(19) United States
(12) Patent Application Publication
(10) Pub. No.: US 2015/0191751 A1
(43) Pub. Date: Jul. 9, 2015

(54) Zero-waste method for producing ethanol and apparatus for implementing same

(71) Applicants: Ajrat Enikeev, Moscow (RU); Valerij Galynkin, St. Petersburg (RU)

(72) Inventors: Ajrat Enikeev, Moscow (RU); Valerij Galynkin, St. Petersburg (RU)

(21) Appl. No.: 14/417,378

(22) PCT Filed: Jul. 26, 2012
(86) PCT No.: PCT/RU2012/000620

§ 371 Date: Jan. 26, 2015

(51) Int. Cl. C12P 7/14; C05F 5/00; C12M 1/02; C12M 1/00; C12M 1/33

(52) U.S. Cl. CPC C12P 7/14; C12M 45/04; C12M 45/09; C12M 45/02; C12M 47/10; C12M 27/00; C12M 47/16; C12M 47/14; C12M 29/04; C05F 5/008; C12P 2201/00

(57) ABSTRACT

A wasteless method for producing ethanol comprises grain milling, mixing same with water in mashing, heat-treating mixed mass accompanied by two-stage enzymatic hydrolysis, sterilizing hydrolyzed mass, cooling same, performing enzymatic vacuum saccharification of hydrolyzed mass, cooling resulting wort down to fermentation temperature, separating wort into first liquid and first solid phases, additionally separating first solid phase into second solid and second liquid phases, fermenting liquid phases, and distilling ethanol from fermented wort.

Second solid phase is dried and used for feed and food production, still bottom from distilling is returned to mashing and also used for irrigation.

A plant for implementing the method comprises receiving hopper, grain separator, hammer mill, vibrating screen, mixer, first and second stage mechanic and enzymatic treatment unit for carbohydrate hydrolysis, sterilizer, vacuum saccharifier, at least one pre-thickener including hydrocyclone or decanter centrifuge, additional separator, drying apparatus, and ultrafiltration unit connected to rectification unit distilling ethanol.

### CROSS-REFERENCE TO RELATED APPLICATIONS

This application is a U.S. CIP National phase application of an International application PCT/RU2012/000620 filed on Jul. 26, 2012, the International application being hereby incorporated by reference in its entirety.

### BACKGROUND OF THE INVENTION

1. Field of the invention

The invention relates to alcohol and pharmaceutical technologies and, more specifically, to comprehensive methods of producing ethanol without distillery grains, as well as obtaining feed stuff and food grain product for the preparation of remedial bakery (zehrnolite).

2. Description of the related art

Ethanol (an INN (International Nonproprietary Name)) currently produced for the pharmaceutical industry needs is a 95% solution used as an antiseptic and disinfectant (manufacturer's monograph 42-0252-5892-04). Process flow chart describing the production thereof corresponds to a method of producing ethanol from starch-containing raw materials.

Various technical solutions of producing ethanol are known. For example, known is a method of producing ethanol from grain raw (RF 2192469, IPC C 12P7/06, pub. Nov. 10, 2002). The method comprises preparing batch at liquor ratio 1:1.8-2.5 and at 50-70° C. with simultaneous dispersion, subjecting the batch (pulp) to water-heat and enzymatic treatment at vigorous stiffing at 75-90° C. for 25-30 min, recycling 20-30% of the mass to the batch preparation, subjecting the remaining part to extensive enzymatic hydrolysis of starch to a concentration of 24-28% of solids in the wort at 55-58° C. for 2-4 hours, fermenting saccharified wort for 36-40 hours, to thereby obtain ethanol and distillers grains, distillers grains being divided into a dispersed phase used for producing feed stuff and a liquid phase, a portion of which, in the amount of 30-40% of the total process water, is recycled to the batch preparation, and the rest is directed to the stage of the fermentation of the saccharified wort for diluting same.

Also known is a method of comprehensive processing of raw grain into alcohol and forage product (RF 2396007, IPC A23K1/165, pub. Aug. 10, 2010). The invention relates to biotechnology as applied to alcohol and feedstuff. The method includes comprehensive processing of raw grain into alcohol and forage products enriched with lysine, based on bioconversion of grain polymers. The comprehensive processing of raw grain is carried out by exogenous enzymes that catalyze hydrolysis of starch, non-starch polysaccharides and proteins. The enzyme-treated cereal wort is used to produce ethanol. Alcohol is distilled, and distillery grains are separated into dispersion and liquid portions. The disperse portion of the distillery grains is dried. The liquid portion of the distillery grains is used for diluting, in the ratio of 1:2-1:10, grain wort take-off from alcohol production for making medium to produce feedstuff. Mineral salts and growth factors, such as corn steep liquor or yeast hydrolyzate, are added to the nutrient medium, the medium is inoculated with the culture-producer, the biosynthesis is carried out under aerobic conditions at 30° C. until the maximum accumulation of the desired product containing amino acids and protein in a culture fluid.

Also known is a Russian patent application No. 93035904 for “A method of producing ethanol from raw grain. Essentially, the method is as follows. Grain is milled and fractionated on husk and endosperm, each fraction is mixed with water and subjected to water-heat treatment. The fraction containing the husk is heated at 150-160° C. for 1-1.5 h, then cooled to 60° C., and cellulolytic enzymes in the form of the fungus culture or the culture fluid and amylolytic enzymes are added to thereby perform hydrolysis of starch, cellulose and other non-starch carbohydrates over 3-4 hours. Endosperm is also mixed with water, subjected to water-heat treatment at 65-75° C. for 1-2.5 h, amylolytic enzymes are added to perform hydrolysis of the starch, and then it is sterilized at 85-95° C. and kept at this temperature for 35-45 minutes. After the hydrolysis, the endosperm is mixed with the husks and is fed to an additional hydrolysis (saccharification) by amylolytic enzymes. An additional hydrolysis is carried out for a mixture of endosperm with husk, or endosperm and husk are hydrolyzed separately and then are mixed. After the primary enzymatic hydrolysis of husk, the resulting wort can be divided into two parts: solid and liquid, the latter is recycled to the water-heat treatment and enzymatic hydrolysis of the endosperm, and the solid part is added to the distillery grains and used as a feed product.

The drawback of all the above methods is that a large amount of production waste, namely liquid distillery grains, is formed in the process of producing ethanol from grain raw materials, the waste containing toxic components (furfural, methanol, fusel aldehydes), polluting environment when discharged into a runoff. In all above-described methods, the solid fraction, which consists of residues of grain, passes through all the stages of the production of ethanol and presents ballast. During fermentation, the precipitate sorbs yeast that prolongs the process by 8-12 hours. Besides, toxic substances are formed from the grain components during the fermentation process; also, during rectification, solid phase scores plates, that results in a shutdown of the rectification and cleaning the column from sediment.

The closest to the proposed method by essential features is a method of producing ethanol from raw grain according to for RF patent No. 2127760, IPC C12P7/06, pub. Mar. 20, 1999. The prior art method involves separating grains from the husk, milling the grains separated from the husk, mixing the grains with a liquid fraction, heat treating the mixture with enzymatic hydrolysis by amylolytic enzymes, sterilizing the mass, cooling same, subjecting the mass to additional enzymatic hydrolysis-saccharification, cooling same to a temperature of fermentation, fermenting obtained wort in a continuous in-line process in a battery of fermenters, to thereby obtain alcohol and distillery grains, adding the separated husk to the distillery grains for use as a feedstuff, characterized in that a portion of obtained distillery grains is set apart before adding the husk thereto and is separated into two streams, the first stream being directed back to the stage of heat treatment, mixing same with water and the milled grain separated from the husk, and the other stream being directed back to the stage of fermentation, dividing same into flows according to the number of the fermenters in the battery and topping the fermenting medium thereby in each fermenter in the amount of 15-20% of the volume of the wort under fermentation after 15-16 h from the start of the fermentation.

Disadvantages of the prototype are that formed in the manufacturing process is distillery grains comprising toxic components such as furfural, methanol, fusel aldehydes and fusel melanins that slowly hydrolyze to release aldehydes in the air. Swamping occurs, which significantly affects the environment. Toxicity of the distillery grains is 10%, meaning that its use is only possible as a feed additive, not exceeding 10% of the total volume. The use of non-starch polysaccharides (cellulose, hemicellulose) increases propanol in ethanol that does not comply with the standard of ethanol. Furthermore, the occurrence of run-off results in a dramatic deterioration of the environment.

### SUMMARY OF THE INVENTION

The object of the present invention is to develop a method and a plant for waste-free production of ethanol, ensuring an environmentally clean and efficient process thereof.

The technical result of the use of the present method consists in improving environmental friendliness and efficiency of the method and apparatus for producing ethanol, as well as in obtaining an additional grain food product (zehrnolite) for humans and animals.

The technical result is achieved due to the fact that in a wasteless method for producing ethanol, the method comprising the steps of grain milling, mixing same with water, heat-treating mass of the mixture, to subject same to hydrolysis, sterilizing the hydrolyzed mass, saccharifying the mass after sterilizing, cooling a resulting product down to a fermentation temperature, whereby wort is produced, and fermenting the wort to distill ethanol, it is proposed to separate the wort prior to fermenting into a first liquid and a first solid phases, to additionally separate the first solid phase into a second solid phase and a second liquid phases, to dry the second solid phase, to thereby produce a cereal product, to use the first and the second liquid phases as a culture medium in fermenting, and to return in part still bottom resulting from distilling back to the stage of mixing and to use same in part as a mineral feed for crop plants.

An additional feature of the method is that the hydrolysis includes preliminary hydrolysis in the presence of amylolytic enzymes followed by holding and secondary breakdown of carbohydrates.

One more additional feature of the method is that the preliminary hydrolysis comprises heating the mass with enzymes up to 60-75° C. for 2-2.5 hours.

Yet one more additional feature of the method is that after the preliminary hydrolysis, a pre-hydrolyzed mass is jet-steamed up to 70-80° C., and the secondary breakdown of carbohydrates comprises holding the pre-hydrolyzed mass at 60-70° C. for 4-4.5 hours.

Still one more additional feature of the method is that the mass is heated up to 80-90° C. prior to the preliminary hydrolysis.

Also, an additional feature of the method is that the pre-hydrolyzed mass is jet-steamed up to 70-80° C. prior to the secondary breakdown of carbohydrates.

An additional feature of the method is also jet-steaming the hydrolyzed mass up to 100-110° C. prior to sterilizing.

Sterilizing, according to one more additional feature of the method, comprises exposing the hydrolyzed mass to 90-100° C. for about 30 minutes.

Saccharifying, in accordance with yet another additional feature of the method comprises quick-cooling the mass down to 50-60° C. at rarefaction of about 0.08-0.09 MPa and adding glucoamylase enzyme preparation.

According to still another additional feature of the method, the first solid phase has moisture of about 80-90%.

Also in accordance with still yet another feature of the method, the second solid phase has moisture of about 55-60%.

The technical result is also achieved by virtue of providing a plant for waste-free production of ethanol, which comprises a receiving hopper, a grain separator connected to the hopper, a hammer mill receiving grain from the grain separator, a vibrating screen for selecting milled grain, a mixer for mixing the selected milled grain with water into a mass, a mechanic and enzymatic treatment unit of the mass to subject same to hydrolysis, a sterilizer of the hydrolyzed mass, a vacuum saccharifier of the sterilized mass, and rectification units for distilling ethanol, wherein the plant is also provided with connected in sequence at least one pre-thickener for separating solid and liquid phases of the mass provided from the vacuum saccharifier, an additional separator of solid and liquid phases, a drying apparatus and an ultrafiltration unit connected to the rectification units.

An additional feature of the plant is using a hydrocyclone with taper of 10-38° as the at least one pre-thickener.

One more additional feature of the plant is using a druk filter as the at least one pre-thickener.

Yet one more additional feature of the plant is using a decanter centrifuge as the at least one pre-thickener.

Still one more additional feature of the plant is using a suction filter with a metal sieve of 30-50 microns as the at least one pre-thickener.

Also, an additional feature of the plant is using a FAN separator as the additional separator.

As an additional feature, the plant can use a decanter centrifuge as the additional separator.

As one more additional feature, the plant can use an a-Laval separator as the additional separator.

The drying apparatus of the plant can, according to another additional feature, include a “fluidized bed” drying apparatus.

Yet another feature of the plant is that the “fluidized bed” drying apparatus comprises an inert filler.

### BRIEF DESCRIPTION OF DRAWINGS

These and other objects and features of the invention will be better understood from the ensuing description when considered together with accompanying drawings where

FIG. 1 illustrates a first embodiment of the plant for waste-free process for producing ethanol, and

FIG. 2 depicts a second embodiment of the plant for waste-free process for producing ethanol.

### DETAILED DESCRIPTION

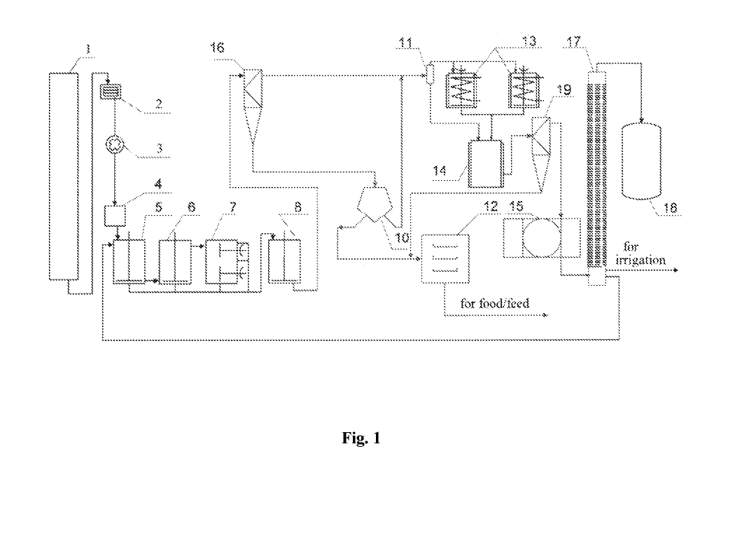

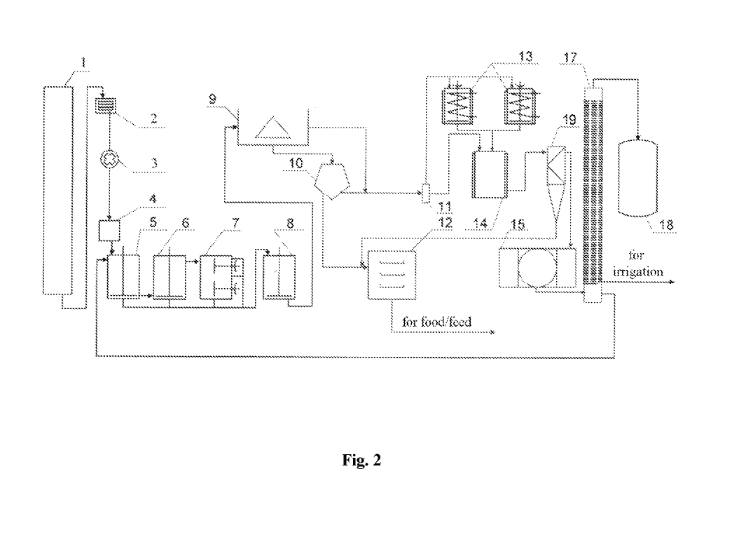

The plants shown in FIGS. 1 and 2 comprise a receiving hopper 1 feeding a grain separator 2 which supplies grain to a hammer mill 3 connected to a vibrosieve 4 from which a product goes to a mixer 5. Also in the plants are a two-stage mechanic and enzymatic treatment (TSMET) unit 6 fed from the mixer 5, and a sterilizer 7 at the output of the unit 6 and connected to a vacuum saccharifier 8. The output of the vacuum saccharifier 8 is connected to a decanter centrifuge 9 (FIG. 2) attached to a solid/liquid phase separator 10 coupled to a distribution head 11 and to a drying apparatus 12. Through the head 11, the centrifuge 9 and separator 10 join a fermenter 14 directly or via a seeding apparatus (inoculators) 13. The fermenter 14 is connected to a hydrocyclone 19, which in turn feeds the drying apparatus 12 and an ultrafiltration unit 15 connected to a distillation tower 17 draining ethanol to a receiver 18. In a processing chain of FIG. 1, a hydrocyclone 16 is put instead of the decanter centrifuge 9 of FIG. 2.

In operation, grain from a hopper 1 is fed to the grain a separator 2, then it goes to the hammer mill 3 and a vibrosieve 4, the screening from the vibrosieve 4 being directed back to the a hammer mill 3. The milled raw material is supposed to be able to pass through the vibrosieve 4 with the sieve mesh a diameter of 1 mm, the residue on the vibrosieve 4 being no more than 15%, and the residue on the vibrosieve with the sieve mesh diameter of 3 mm is not supposed to exist. Further, the grain powder is fed to the mixer 5 where it is mixed with a liquid fraction (water) at 50-55° C. Then, amylolytic enzymes are added to the mixture, and it is warmed to a temperature of 80-90° C., the heated mass being then supplied to the TSMET unit 6 where the first stage of TSMET, namely, hydrolysis of carbohydrates, takes place during 2-2.5 hours at 60-75° C. After a holding, the mass is jet-steamed to the temperature of 70-80° C. and is subjected to the second stage of TSMET, where a deeper breakdown of carbohydrates takes place at 60-70° C. during 4-4.5 hours. Thus, a preliminary enzymatic hydrolysis is carried out in the course of the water-heat treatment.

Further, the softened and hydrolyzed mass is jet-steamed to the temperature of 100-110° C. and is fed to the sterilizer 7, where it is maintained for sterilization at 90-100° C. for about 30 minutes. From the sterilizer 7, the mass enters the vacuum saccharifier 8 where quick cooling of the mass from the temperature of 100° C. to 50-60° C. at rarefaction of 0.08-0.09 MPa and saccharification by feeding a glucoamylase enzyme preparation occur. The cooled mass is kept for 1-2 hours, and is further cooled to a fermentation temperature. Then, the resulting wort with solid residue (grain leftover after heat and enzymatic treatment) enters a pre-thickener, which includes the hydrocyclone 16 or a decanter centrifuge 9. There, a partial separation of the liquid and solid phases takes place. The solid phase with 80-90% moisture is fed to the separator 10 (FAN separator), where the solid phase with moisture of 55-60% is formed and fed to the drying apparatus 12. Appearing from the drying apparatus 12 is dry zehrnolite, representing a ready-grain product, which can be stored for much longer than whole grains. After the separation, the liquid phase (wort) is supplied to the distribution head 11 and further goes to the seeding apparatus (inoculators) 13 as a medium, as well as to the fermenter 14 for the fermentation stage in ethanol distillation. After the fermentation, the liquid phase is fed to the hydrocyclone 19 serving for pre-cleaning the culture fluid to get rid of suspended matter and thus avoid clogging the ultrafiltration unit 15, where proteins and polysaccharides are removed, and to the distillation tower 17 and receiver 18. The aqueous phase (still bottom) containing mineral components is directed back to the mashing step (mixer 5), and the rest may be used for irrigating farmland.

During the separation by the hydrocyclone 16 (for which a conventional unit of the type shown and described at http://en.wikipedia.org/wiki/Hydrocyclone can be used, the taper of conical part of 10-38° being preferable therefor), the slurry that is being separated is pumped under the pressure of no less than 0.2 MPa through the pipe, attached tangentially to the cylindrical part of the hydrocyclone (not shown). In the helical motion of the suspension, the solid particles thereof, are centrifugally thrown against the walls of the conical part (not designated in the drawings) of the a hydrocyclone 16, fall down and are removed into a receiver (not shown). The internal flow of the clarified liquid phase gyrates along the axis of the hydrocyclone 16 counter to the external flow and is output to another a receiver (not shown). The effectiveness of the separation of the slurry depends significantly on the ratio of the diameter of the lower outlet pipe (not shown) to the diameter of the pipe for discharging the clarified liquid phase (not shown).

Used at this stage may be a decanter centrifuge 9 that provides an effective solution of the problem of dewatering the sludge formed. The decanter centrifuge 9 allows minimizing the amount of waste and allows using water for recycling. Instead of the hydrocyclone 16, a suction filter (not shown) with a metal sieve of 30-50 microns or a pressure (druk) filter (not shown) or a vacuum drum filter (not shown) can be used as the pre-thickener.

The separator 10 can include a FAN PSS 3.2-1040 separator or the decanter centrifuge 9 or an a-Laval separator, that is filled by a pump or by gravity from a storage tank. The separator 10 can be fed from a hopper. The optimal loading option depends on local conditions and the consistency of the raw material.

Essentially, the heat treatment process in the drying apparatus 12 of the “fluidized bed” type is as follows. Upon airing a layer of the loose granular product placed on a gas distribution grid by a drying agent (hot air), the product rises into a half-fluidized state and acquires flow properties. In this state, the layer is loosened and stirred vigorously, so that all the material particles are uniformly acted upon by the drying agent. Due to this stirring and mutual contact of the individual particles, temperature equalization in the layer volume occurs, which is especially important when drying thermolabile products. It is noteworthy that due to the peculiarities of the specified process, the drying efficiency and qualitative indicators of processed products achieved in fluidized bed installations are considerably higher than in conventionally used drum, auger, belt or tunnel dryers. The drying apparatus 12 operates under rarefaction and is provided with appropriate seals. The air is filtered before being fed to the drying apparatus 12. Drying takes place at a constant temperature and high speed. Standard drying time is 20-30 minutes. It is possible to use the “fluidized bed” drying apparatus 12 with an inert filler, on which a dry cereal product in the form of granules is obtained.

Examples of the proposed waste-free process for producing ethanol using the proposed plant are as follows.

### EXAMPLE 1

Corn (initial parameters: protein ˜12%; starch ˜67%), is pre-milled, mixed with a liquid fraction at a temperature of 50-55° C. Amylolytic enzymes are introduced into the mash, it is heated up to 80-90° C., and a preliminary enzymatic hydrolysis is carried out in the process of water-heat treatment. The mass is then sterilized at 100-110° C., cooled down to 56-58° C. and held for 1-2 hours. As this takes place, an enzyme complex is added, and the final hydrolysis (saccharification) is carried out. The resulting wort with solid residue (left after the heat and enzymatic treatment) enters a hydrocyclone, where a partial separation of the liquid and solid phases occurs. The solid phase having moisture of 85-90% is applied to a separator, where the solid phase at moisture of 55-60% is received and fed to a drying apparatus whereby dry zehrnolite is obtained.

Liquid phases after the hydrocyclone and after the separator are combined and are further used as a medium for the fermentation stage in the production of ethanol (or for the cultivation of other microorganisms, such as baker's yeast (Saccharomyces cerevisiae)). After the fermentation, the liquid phase is fed to the hydrocyclone, ultrafiltration unit, and rectification units for distillation of ethanol, the aqueous phase containing mineral components may be returned to the mashing step, and the rest part may be used for irrigation of farmland.

Fermented wort strength is 8%.

Dry residue—317 g of grain product is obtained out of 11 liters.

### EXAMPLE 2

A mixture of rye and wheat grain is pre-milled, mixed with the liquid fraction at the a temperature of 50-65° C. Amylolytic enzymes are added to the mash, which is heated up to 80-90° C., and the preliminary enzymatic hydrolysis is carried out in the process of the water-heat treatment. The mass is then sterilized at 100-110° C., cooled down to 50-60° C. and held for 1-2 hours. As this takes place, an enzyme complex is added, and the final hydrolysis is carried out (saccharification). The resulting wort having a solid sediment (grain residue after the heat and enzymatic treatment) enters a decanter centrifuge (a suction filter, a pressure filter), where a partial separation of liquid and solid phases occurs. The solid phase with moisture content of 80-90% is applied to a separator, where the solid phase with moisture of 50-60% results therefrom and is fed to a drying apparatus. Whereby, dry forage (feed) and food product “zehrnolite” is obtained.

The liquid phases after the decanter centrifuge and a separator are combined and are further used as a medium for the inoculators and for the fermentation stage in the production of ethanol. After the fermentation, the liquid phase is fed to the hydrocyclone, ultrafiltration unit and rectification units for distillation of ethanol, and the aqueous phase containing mineral components may be returned to the mashing step, and the rest part may be used for irrigation of farmland.

Fermented wort strength is 8%.

Dry residue—384 g of grain product is obtained out of 11 liters.

### EXAMPLE 3

### (Performing the Process According to Manufacturing Technology).

Corn (initial parameters: protein ˜12%; starch ˜67%) is milled and mixed with the liquid fraction at the temperature of 50-55° C. Amylolytic enzymes are added to the mash, which is heated up to 80-90° C., and the preliminary enzymatic hydrolysis is carried out in the process of the water-heat treatment. The mass is then sterilized at 100-110° C., cooled down to 50-60° C., and held for 1-2 hours. As this takes place, an enzyme complex is added, and the final hydrolysis of the mass is carried out (saccharification), and it is further cooled to the fermentation temperature. The resulting wort having a solid sediment (grain residue after the heat and enzymatic treatment) is supplied to the fermentation step (fermentation). After fermentation, the fermented wort is fed to the hydrocyclone, ultrafiltration unit, and distillation towers (rectification units) where a separation of the culture fluid into ethanol and heavy fraction (distillery grain) occurs. After washing, the heavy fraction is sent to a heavy fraction collector, and from there—to a storage. The distillery grain is then used as a farmland fertilizer and animal feed.

Fermented wort strength is 8.7%.

Dry residue of distillery grain—4.7%.

### EXAMPLE 4

A mixture of rye and wheat grain is milled, mixed with water (30% of the volume) and aqueous phase (70% of the volume) containing mineral components (obtained after the distillation step) at a temperature of 50-55° C. Amylolytic enzymes are added to the mash, which is heated up to 80-90° C., and the preliminary enzymatic hydrolysis is carried out in the process of water-heat treatment. The mass is then sterilized at 100-110° C., cooled down to 50-60° C., and held for 1-2 hours. As this takes place, an enzyme complex is added, and the final hydrolysis (saccharification) of the mass is carried out, and it is further cooled to the fermentation temperature. The resulting wort with solid residue (grain residue after heat and enzymatic treatment) enters a hydrocyclone, where a partial separation of liquid and solid phases takes place. The solid phase with moisture content of 75-90% is applied to a separator, where the solid phase with moisture of 50-60% results therefrom and is fed to the drying apparatus. In such a way, dry “zehrnolite” is obtained.

Liquid phases after the hydrocyclone and separator are combined and fed to an ultrafiltration unit. They are further used as a medium for the fermentation stage in the production of ethanol (or for the cultivation of other microorganisms). After the fermentation, the liquid phase is fed to the hydrocyclone and ultrafiltration unit, and then—to rectification units for distillation of ethanol. The remaining aqueous phase containing mineral components is returned to the mashing step, and the rest part is used for farmland irrigation.

Fermented wort strength is 8%.

Dry residue—390 g of grain product is obtained out of 11 liters.

Thus, the invention has the following advantages over conventional technologies:
high yield of ethanol in the fermentation of liquid wort;
complete absence of distillery grain;
obtaining additional feed and food products;
environmentally friendly technology;
separation of the solid fraction before the fermentation stage allows applying liquid fraction to rectification units after fermentation, thus preventing fouling rectifying plates by proteins and polysaccharides from occurring. As a result, rectification units can work for at least 2-3 months non-stop. In currently used technologies, rectification units are to be shut down every 5-6 days to clean the plates of the components fouled thereon;
the still bottom—after the distillation of ethanol in the en rectification units—represents an aqueous solution of mineral components that can be re-used at the slurry preparation step with the milled grain and for farmland irrigation;
returning water to the cycle allows reducing its consumption in the process and reduce the amount of liquid waste in the production by 2-3 times.
providing a cereal product with a 50% toxicity level, meaning that its portion as a base for remedial (medical) baked goods of therapeutic purpose or feed for cattle and poultry can reach up to 50% of the total volume.

1-15. (canceled)

16. A wasteless method of producing ethanol, the method comprising the steps of:
milling grain,
mixing same with water,
heat-treating mass of the mixture subjecting same to hydrolysis,
sterilizing the hydrolyzed mass,
saccharifying the mass after the sterilization,
cooling a resulting product down to a fermentation temperature, whereby wort is produced, and
fermenting the wort to distill ethanol,
wherein prior to said fermenting:
said wort is separated into a first liquid and a first solid phases,
said first solid phase is additionally separated into a second solid phase and a second liquid phases, and
said second solid phase is dried, to thereby produce a cereal product, whereas
said first and said second liquid phases are directed to said fermenting and are also used as a culture medium therefor, and
wherein still bottom resulting from said distilling is in part returned to said stage of mixing and in part is used as a mineral feed for crop plants.

17. The method according to claim 16, wherein said hydrolysis includes preliminary hydrolysis in the presence of amylolytic enzymes followed by holding and secondary breakdown of carbohydrates.

18. The method according to claim 17, wherein said preliminary hydrolysis comprises heating the mass with enzymes up to 60-75° C. for 2-2.5 hours.

19. The method according to claim 17, wherein after said preliminary hydrolysis, a pre-hydrolyzed mass is jet-steamed up to 70-80° C., and said secondary breakdown of carbohydrates comprises holding said pre-hydrolyzed mass at 60-70° C. for 4-4.5 hours.

20. The method according to claim 17, wherein said mass is heated up to 80-90° C. prior to said preliminary hydrolysis.

21. The method according to claim 17, wherein said pre-hydrolyzed mass is jet-steamed up to 70-80° C. prior to said secondary breakdown of carbohydrates.

22. The method according to claim 16, wherein said hydrolyzed mass is jet-steamed up to 100-110° C. prior to said sterilizing.

23. The method according to claim 16, wherein said sterilizing comprises exposing said hydrolyzed mass to 90-100° C. for about 30 minutes.

24. The method according to claim 16, wherein said saccharifying comprises quick-cooling the mass down to 50-60° C. at rarefaction of about 0.08-0.09 MPa and adding a glucoamylase preparation.

25. The method according to claim 16, wherein said first solid phase has moisture of about 80-90%.

26. The method according to claim 16, wherein said second solid phase has moisture of about 55-60%.

27. The method according to claim 16 wherein the cereal product is used as a basis for bakery products of therapeutic purpose.

28. The method according to claim 16 wherein the cereal product is used as feed for cattle and poultry.

29. A plant for waste-free production of ethanol, the plant comprising:
a receiving hopper,
a grain separator connected to the hopper,
a hammer mill receiving grain from the grain separator,
a vibrating screen for selecting milled grain,
a mixer for mixing the selected milled grain with water into a mass,
a mechanic and enzymatic treatment unit of the mass to subject same to hydrolysis,
a sterilizer of the hydrolyzed mass,
a vacuum saccharifier of the sterilized mass, and
rectification units for distilling ethanol,
wherein the plant is provided with connected in sequence at least one pre-thickener for separating solid and liquid phases of the mass supplied from the vacuum saccharifier, an additional separator of solid and liquid phases, a drying apparatus and an ultrafiltration unit connected to the rectification units.

30. The plant according to claim 29, wherein the at least one pre-thickener includes a hydrocyclone with taper of 10-38°.

31. The plant according to claim 29, wherein the at least one pre-thickener includes a suction filter with a metal sieve of 30-50 microns.

32. The plant according to claim 29, wherein the at least one pre-thickener includes a druk filter.

33. The plant according to claim 29, wherein the at least one pre-thickener includes a decanter centrifuge.

34. The plant according to claim 29, wherein the at least one pre-thickener includes vacuum drum filter.

35. The plant according to claim 29, wherein the additional separator includes a FAN separator.

36. The plant according to claim 29, wherein the additional separator includes a decanter centrifuge.

37. The plant according to claim 29, wherein the additional separator includes an α-Laval separator.

38. The plant according to claim 29, wherein the drying apparatus includes a “fluidized bed” drying apparatus.

39. The plant according to claim 38, wherein the “fluidized bed” drying apparatus is provided with an inert filler.
